(19) United States
(12) Patent Application Publication
(10) Pub. No.: US 2025/0275849 A1
(43) Pub. Date: Sep. 4, 2025

(54) THORACOAORTIC HYBRID GRAFT AND SURGICAL METHODS THEREOF

(71) Applicant: THE COOPER HEALTH SYSTEM, A NEW JERSEY NON-PROFIT CORPORATION, Camden, NJ (US)

(72) Inventor: Joseph V. Lombardi, Turnersville, NJ (US)

(73) Assignee: THE COOPER HEALTH SYSTEM, A NEW JERSEY NON-PROFIT CORPORATION, Camden, NJ (US)

(21) Appl. No.: 18/862,613

(22) PCT Filed: May 2, 2023
(86) PCT No.: PCT/US2023/020710

§ 371 Date: Nov. 4, 2024

Related U.S. Application Data
(60) Provisional application No. 63/337,581, filed on May 2, 2022.
(60) Provisional application No. 63/339,287, filed on May 6, 2022.

(51) Int. Cl. A61F 2/07; A61F 2/06; A61F 2/848; A61M 60/117; A61M 60/35

(52) U.S. Cl. CPC A61F 2/07; A61M 60/117; A61M 60/35; A61F 2002/065; A61F 2002/8483; A61F 2250/001

(57) ABSTRACT

A surgical kit for repairing an aortic aneurysm comprising a vascular prosthesis with a primary conduit forming a primary lumen extending from a primary inlet opening to a primary outlet opening, and a perfusion branch conduit forming a perfusion branch lumen extending from a perfusion branch opening in the primary conduit to a perfusion outlet opening at a distal end of the perfusion branch conduit; and a perfusion apparatus comprising an inlet conduit having a proximal end configured to be fluidly coupled to the distal end of the perfusion branch conduit, a plurality of secondary conduits branching from the inlet conduit and configured for fluid coupling to aortic branches, and a valve assembly configured to selectably alter the plurality of secondary conduits between an open flow state and a closed sealed state.

### CROSS-REFERENCE TO RELATED APPLICATIONS

This application claims the benefit of U.S. Provisional Patent Application No. 63/337,581, filed May 2, 2022, and U.S. Provisional Patent Application No. 63/339,287, filed May 6, 2022. The disclosures of the aforementioned priority applications are incorporated herein by reference in their entireties.

### BACKGROUND

Surgical repairs involving stents have become the standard when treating aortic aneurysms and recent advances have led to the use of hybrid stent-grafts. While use of such technology has proven revolutionary in treating aneurysms in the aortic arch, it has been harder to utilize the technology in repairing the more invasive thoracoabdominal aortic aneurysms. In addition, surgeons are still often required to utilize heart bypass techniques which increase the difficulty of successfully performing these already arduous surgical repairs. Thus, there is a need in the art for a hybrid stent-graft with an attached perfusion apparatus that allows a surgeon to repair a thoracoabdominal aneurysm while minimizing the need for a heart bypass.

### BRIEF SUMMARY

One aspect of the present invention may be directed to a surgical kit for repairing an aortic aneurysm and components thereof. In one embodiment the surgical kit may comprise a vascular prosthesis comprising a primary lumen extending from a primary inlet opening to a primary outlet opening, the primary lumen configured to be fluidly coupled to the aorta of a patient; and a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen. In some embodiments, the vascular prosthesis further comprises a plurality of branch conduits, each of the branch conduits forming a branch lumen extending from an opening in the primary lumen to a branch outlet opening, the branch inlet opening in fluid communication with the primary lumen, and a distal end of each of the branch conduits configured to be fluidly coupled to an aortic branch

In some embodiments, the surgical kit further comprises a perfusion apparatus comprising an inlet conduit having a proximal end configured to be fluidly coupled to the distal end of the perfusion branch conduit; a plurality of secondary conduits branching from the inlet conduit and configured for fluid coupling to aortic branches; and a valve assembly configured to selectably alter the plurality of secondary conduits between an open flow state and a closed sealed state.

In one embodiment, the primary conduit of the vascular prosthesis comprises a stent-graft portion and a tube-graft portion that meet at a junction region, the stent-graft portion comprising the primary inlet opening and the tube graft portion comprising the primary outlet opening. In certain embodiments, the stent-graft portion comprises a flexible biocompatible membrane attached to a support structure, the stent-graft portion configured to be capable of being radially expanded from a collapsed state to a deployed state.

In some embodiments, the surgical kit further comprises a package containing vascular prosthesis, the perfusion apparatus, and the valve assembly.

In some embodiments, the distal end of the perfusion branch conduit comprises a first quick connect feature and the proximal end of the inlet conduit of the perfusion apparatus comprises a second quick connect feature configured to be matable with the first quick connect feature to fluidly couple the perfusion branch lumen and a passageway of the inlet conduit. In some embodiments, the first and second quick connect features are selected from a group consisting of bayonet connector component, a threaded connector component, a snap-fit connector component, an interference fit connector component, and combinations thereof.

Another aspect of the present invention may be directed to a vascular prosthesis comprising: a primary conduit forming a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; a plurality of branch conduits, each of the branch conduits forming a branch lumen extending from a branch inlet opening in the primary lumen to a branch outlet opening, the branch inlet opening in fluid communication with the primary lumen, and a distal end of each of the branch conduits configured to be fluidly coupled to an aortic branch; and a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen, and the distal end of the perfusion branch conduit comprising a first quick connect feature.

In some embodiments, the first quick connect feature is selected from a group consisting of bayonet connector component, a threaded connector component, a snap-fit connector component, an interference fit connector component, and combinations thereof. In another embodiment, the perfusion branch opening is located closer to the primary inlet opening than any of the branch conduits.

One aspect of the present invention may be directed to a vascular prosthesis comprising: a primary conduit comprising a primary lumen, extending from a primary inlet opening to a primary outlet opening; a plurality of branch inlet openings; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

Another aspect of the present invention may be directed to a vascular prosthesis comprising: a primary conduit comprising a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; four branch conduits, each of the four branch conduits forming a branch lumen extending from a branch inlet opening in the primary conduit to a branch outlet opening, the branch inlet opening in fluid communication with the primary lumen, and a distal end of each of the four branch conduits configured to be fluidly coupled to an aortic branch; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

In yet another aspect, the present invention may be directed to a method of repairing an aortic aneurysm, the method comprising: fluidly coupling: (i) a proximal end of a primary lumen of a vascular prosthesis to a first portion of an aorta of a patient upstream of the aortic aneurysm; and (ii) a distal end of the primary lumen of the vascular prosthesis to a second portion of the aorta downstream of the aortic aneurysm, wherein one or more aortic branches of the patient are located between the first and second portions of the aorta, thereby allowing blood to flow through the primary lumen; fluidly coupling an inlet conduit of a perfusion apparatus to a distal end of a perfusion branch conduit of the vascular prosthesis, a valve assembly of the perfusion apparatus being in a closed sealed state such that blood from the primary lumen of the vascular prosthesis cannot flow through a plurality of secondary conduits of the perfusion apparatus that branch from the inlet conduit; fluidly coupling distal ends of the secondary conduits to the one or more aortic branches; and altering the valve assembly to an open flow state to restore blood flow to the one or more aortic branches.

### BRIEF DESCRIPTION OF THE DRAWINGS

The detailed description of the invention will be better understood when read in conjunction with the appended drawings. It should be understood, however, that the invention is not limited to the precise arrangements and instrumentalities of the embodiments shown in the drawings.

FIG. 1 illustrates a surgical kit in accordance with one embodiment of the present invention;

FIG. 2 illustrates a vascular prosthesis in accordance with one embodiment of the present invention;

FIG. 3 illustrates a cross sectional view of the vascular prosthesis of FIG. 2;

FIG. 4 illustrates a perfusion apparatus in accordance with one embodiment of the present invention;

FIG. 5 illustrates the perfusion apparatus shown in FIG. 4 attached to the vascular prosthesis shown in FIGS. 2 and 3;

FIGS. 6A and 6B illustrate a coupling mechanism between the vascular prosthesis and perfusion apparatus as shown in FIG. 5 in accordance with one embodiment of the present invention;

FIG. 7 illustrates the portion of the aorta that the present invention is intended to be located in accordance with one embodiment of the present invention;

FIGS. 8A through 8M illustrate an associated surgical method in accordance with one embodiment of the present invention;

FIGS. 9A and 9B illustrate a vascular prosthesis in accordance with alternate embodiments of the present invention; and

FIG. 10 illustrates a surgical kit in accordance with one embodiment of the present invention.

### DETAILED DESCRIPTION

The following description of the preferred embodiment(s) is merely exemplary in nature and is in no way intended to limit the invention or inventions. The description of illustrative embodiments is intended to be read in connection with the accompanying drawings, which are to be considered part of the entire written description. In the description of the exemplary embodiments disclosed herein, any reference to direction or orientation is merely intended for convenience of description and is not intended in any way to limit the scope of the present inventions. Relative terms such as “lower,” “upper,” “horizontal,” “vertical,” “above,” “below,” “up,” “down,” “left,” “right,” “top,” “bottom,” “front” and “rear” as well as derivatives thereof (e.g., “horizontally,” “downwardly,” “upwardly,” etc.) should be construed to refer to the orientation as then described or as shown in the drawing under discussion. These relative terms are for convenience of description only and do not require a particular orientation unless explicitly indicated as such. Terms such as “attached,” “affixed,” “connected,” “coupled,” “interconnected,” “secured” and other similar terms refer to a relationship wherein structures are secured or attached to one another either directly or indirectly through intervening structures, as well as both movable or rigid attachments or relationships, unless expressly described otherwise. The discussion herein describes and illustrates some possible non-limiting combinations of features that may exist alone or in other combinations of features. Furthermore, as used herein, the term “or” is to be interpreted as a logical operator that results in true whenever one or more of its operands are true.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise define, all technical and scientific terms used herein have the same meaning as commonly understood to one of ordinary skill in the art to which this invention belongs. As used herein, the term “proximal” and “distal” refer to a location or portion of a device that, when inserted, is upstream or downstream with respect to blood flow.

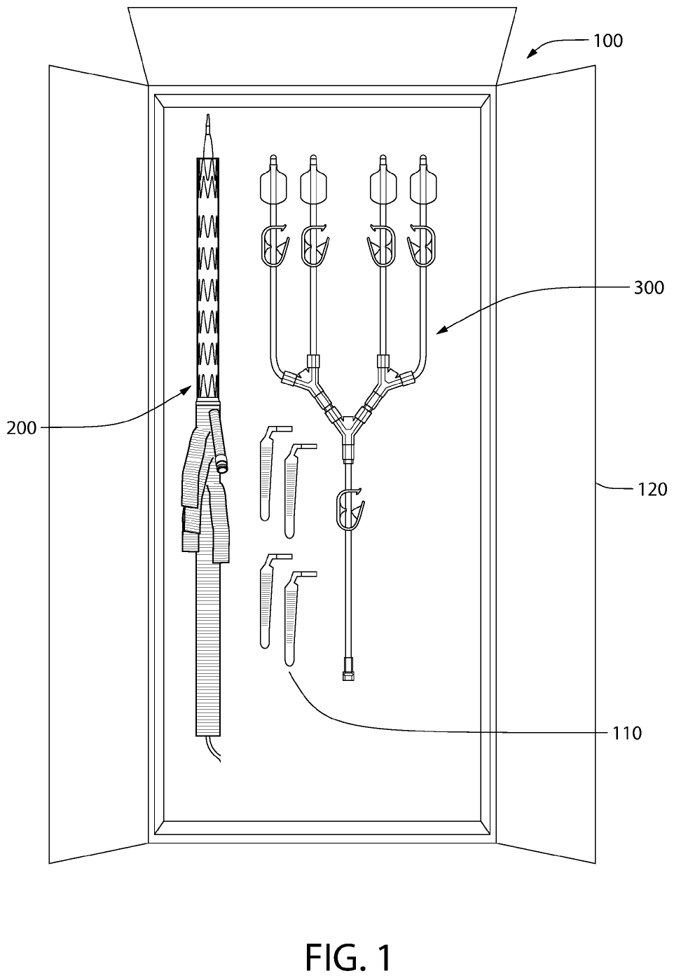

Referring to FIG. 1, a surgical kit 100 is disclosed according to an embodiment of the present invention. The surgical kit 100 generally comprises a vascular prosthesis 200, a perfusion apparatus 300, and at least one surgical accessory 110, all of which are provided in a package 120. The package may be a sealed container that maintains the sterile state of the contents, such as sealed plastic bag, box, or the like.

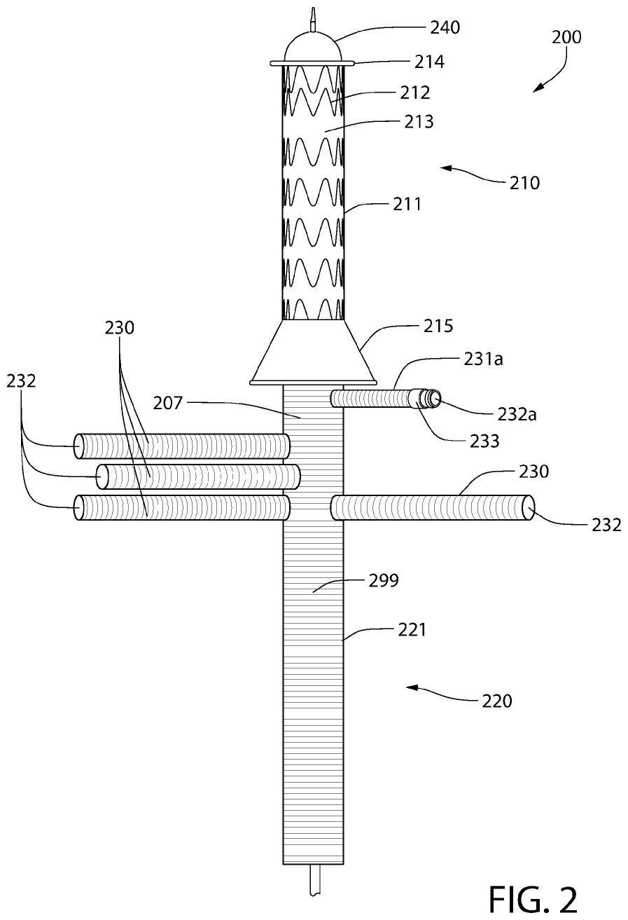

In some embodiments, the surgical kit 100 may further comprise a molding balloon 240 (FIG. 2). The surgical accessories 110 may be tools for cutting, dissecting, suturing, hemostasis, holding, retracting, disinfecting, or soaking. In some embodiments, the surgical accessories 110 may be scalpels, forceps, needles, needle holders, threads, retractors, suction tips, staplers, clips, scissors, hooks, dissectors, hemostats, gauze, saline, trocars, containers, trays, or any combination thereof.

The at least one surgical accessory 110 may be present in any quantity. In a preferred embodiment, the surgical accessories 110 includes at least one clamp 110. While FIG. 1 shows a clamp 110 as the surgical accessory, a person of skill in the art would understand that the surgical kit 100 of the present invention may include other types of surgical accessories. As mentioned above, the surgical kit 100 comprises the package 120 containing the vascular prosthesis 200, the perfusion apparatus 300, and other component, such as the clamps 110. In some embodiments, the package 120 may further comprise the molding balloon 240.

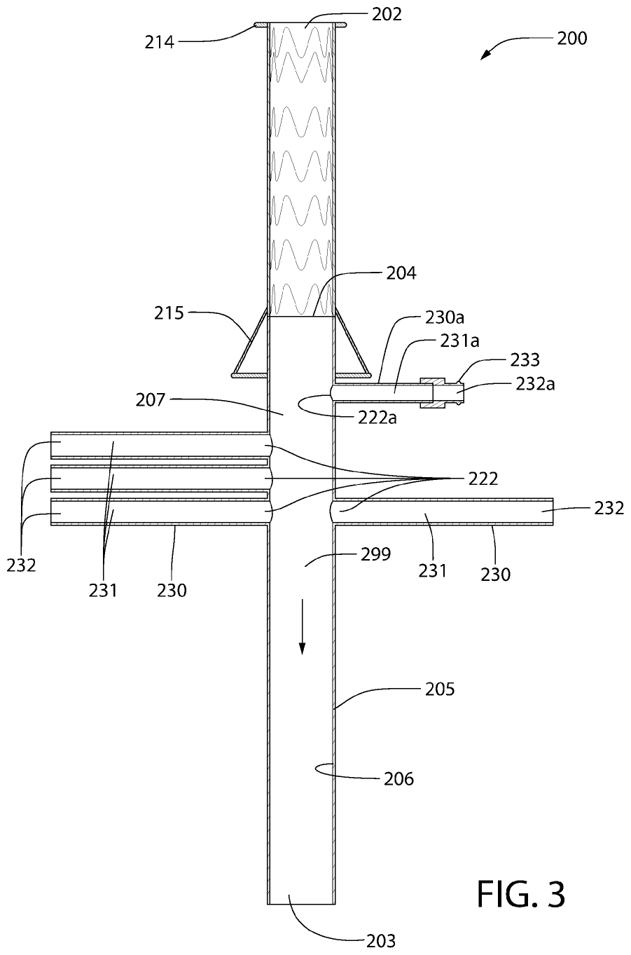

Referring now to FIGS. 2 and 3, the vascular prosthesis 200 will be further described. The vascular prosthesis 200 generally comprises a proximal stent-graft portion 210 and a distal tube-graft portion 220. The vascular prosthesis 200 comprises a primary conduit 299. The primary conduit 299 is a tubular structure having a generally cylindrical transverse cross-sectional profile. The primary conduit 299 has an outer surface 205 and an inner surface 206. The inner surface 206 of the prosthesis 200 defines a primary lumen 201 that extends longitudinally through the vascular prosthesis 200 from a primary inlet opening 202 at the proximal end of the primary conduit 299 to a primary outlet opening 203 at the distal end of the primary conduit 299. The primary conduit 299 is configured to be fluidly coupled to an aorta of a patient to allow fluid such as blood to pass through the primary lumen 201 in a manner similar to the natural lumen of the aorta. As can be seen, a proximal section of the primary conduit 299 is part of the stent-graft portion 210 of the vascular prosthesis 200 while a distal section of the primary conduit 299 is part of the tube graft portion 220. The stent-graft portion 210 of the primary conduit 299 and tube graft portion 220 of the primary conduit 299 are joined together at a junction region 204, which is located between the proximal and distal ends of the primary conduit 299.

The stent-graft portion 210 of the primary conduit 299 is a compressible tube 211 comprising a support structure 212 and a flexible membrane 213. The support structure 212 is configured to provide radial support to the stent-graft portion 210 and to radial expand to conform to the diameter of the surrounding blood vessel when deployed. The support structure 212 may be made of any biocompatible metal, plastic, or derivatives thereof. In some embodiments, the support structure 212 may be made from stainless steel, cobalt chromium, or nitinol. In some embodiments, the support structure 212 may comprise a plurality of support elements. The plurality of support elements may be unconnected, partially interconnected, or fully interconnected. In other embodiments, the support structure 212 may comprise a single support element. The support structure 212 may be a spring, wire, mesh, or any other configuration known in the art. In one embodiment, the support structure 212 is a metal mesh. In some embodiments, the support structure 212 is configured to provide radial support to the stent-graft portion 210.

The membrane 213 is attached to the support structure 212 and configured to be able to radially expand in conjunction with the support structure 212. In some embodiments, the membrane 213 is attached using a thermal process or sewing. The membrane 213 may be made from any flexible biocompatible material that forms a seal. In a preferred embodiment, the membrane 213 is made from woven polyester or derivative thereof. In some embodiments, the membrane 213 may be made from polyester or polyester derivative, polyaramids, polyacrylonitrile, nylon, cellulose, polyurethane, polyethylene terephthalate (PET/Dacron), polytetrafluoroethylene (PTFE), or expanded polytetrafluoroethylene (ePTFE), and thermoplastic polyethylenes such as ultra high molecular weight polyethylene (UHMWPE) or high-modulus polyethylene (HMPE). The membrane 213 may further comprise angiographic markers. The markers may be made from any radiopaque material known in the art. In some embodiments, the angiographic markers may be made from platinum, platinum-iridium, tantalum, gold, or any combination thereof. In some embodiments, the stent-graft portion 210 is comprised solely of a support structure 212 without a membrane 213.

In the exemplified embodiment, the stent-graft portion 210 of the primary conduit 299 comprises a hemostatic collar 214. The hemostatic collar 214 is a flange or ring-like structure that protrudes radially from the proximal end of the primary conduit 299. The hemostatic collar 214 is exemplified as being located at the proximal end of the primary conduit 299 but in other embodiments may be spaced therefrom. The hemostatic collar 214 can be made from any biocompatible material known in the art. In some embodiments, the hemostatic collar 214 is made from biocompatible plastic, metal, or any derivative thereof. In a preferred embodiment, the hemostatic collar 214 is made from biocompatible felt such as polyglycolic acid felt (PGA) or PTFE felt. The hemostatic collar 214 is configured to secure the proximal end of the prosthesis 200 within the aorta. In some embodiments, the hemostatic collar 214 may further comprise interspersed projections or barbs which are configured to prevent the vascular prosthesis 200 from migrating.

In some embodiments, the stent-graft portion 210 may also comprise a hemostatic apron 215. The hemostatic apron 215 is located near the junction region 204. In the exemplified embodiment, the hemostatic apron 215 traverses the junction region 204 such that an upper portion of the hemostatic apron 215 is located above the junction region 204 and a lower portion of the hemostatic apron 215 is located below the junction region 204. The hemostatic apron 215 is connected to the primary conduit 299 above the junction region 204, and extends downwards and outwards to form a generally truncated cone-shaped flange. The hemostatic apron 215 may be made from any biocompatible material known in the art. The apron 215 is configured to be attached to the wall of an aorta in a patient to create an anastomosis between the prosthesis 200 and the aorta to prevent intercostal back bleeding.

The tube graft portion 220 of the primary conduit 299 is a generally cylindrical tube 221 which may comprise a plurality of inlet openings 222 (the purpose of which is discussed in greater detail below). The tube graft portion 220 may be made from any biocompatible material known in the art. In some embodiments, the tube graft portion 220 may be made from polyester or polyester derivative, polyurethane, polyethylene terephthalate (PET/Dacron), polytetrafluoroethylene (PTFE), or expanded polytetrafluoroethylene (ePTFE), thermoplastic polyethylenes such as ultra high molecular weight polyethylene (UHMWPE) or high-modulus polyethylene (HMPE). In a preferred embodiment, the tube graft portion 220 is made from polyethylene terephthalate (PET/Dacron).

The plurality of openings 222 fluidly connect the primary lumen 201 to a plurality of branch lumens 231, which are formed by the branch conduits 230. The plurality of openings 222 may be referred to as branch inlet openings 222 when being discussed in relation to the branch conduits 230. In the exemplified embodiment, the vascular prosthesis 200 further comprises a plurality of branch conduits 230. Each of the branch conduits 230 forms a branch lumen 231 that extends from one of the openings 222 at the proximal end thereof to a branch outlet opening 232 at the distal end thereof. The branch lumens 231 are part of the tube graft portion 220 of the vascular prosthesis 200.

While four branch conduits 230 are shown in the exemplified embodiment, more or less branch conduits 230 can be provided as desired and depending on the surgical requirements. Moreover, in certain embodiment, no branch conduits 230 may be include and only a perfusion branch conduit 230a may be included. When intended to be used to couple to aortic branches of the patient, the branch conduits 230 may be referred to as aortic branch conduits 230 of the vascular prosthesis 200. In certain embodiments, one or more of the branch conduits 230 may be removable or temporary.

The distal end of branch conduits 230 are configured to be fluidly coupled to an aortic branch of a patient. In a preferred embodiment, the branch conduits 230 are configured to be fluidly coupled to visceral arteries. In some embodiments, multiple branch conduits 230 are connected to multiple aortic branches through a single connection. In other embodiments, the branch conduits 230 are connected to aortic branches through separate connections.

The vascular prosthesis 200 further comprises a perfusion branch conduit 230a. To accommodate the perfusion branch conduit 230a the primary conduit 299 comprises a perfusion branch opening 222a in a sidewall thereof. The perfusion branch opening 222a is closer to the proximal end of the primary conduit 299 than any of the openings 222 of the branch conduits 230. The axial section of the primary conduit 299 that is between the perfusion branch opening 222a and the closest of the openings 222 provides a clamping zone 207 so that the primary lumen 201 can be sealed by the application of a clamp to the primary conduit 299 in this axial section (discussed in greater detail below). As exemplified, the perfusion branch conduit 230a is located at the proximal end of the tube-graft portion 220, generally below the junction region 204. The clamping zone 207 is located directly below the perfusion branch conduit 230a and above any other branch conduits 224. The clamping zone 207 is an axial section of the tube-graft portion 220 of the primary conduit 299 that lacks any openings 222.

The perfusion branch conduit 230a forms a perfusion branch lumen 231a which extends from the perfusion branch opening 222a at the proximal end of the perfusion branch conduit 230a to a perfusion outlet opening 232a at the distal end of the perfusion branch conduit 230a. The perfusion branch opening 222a fluidly connects the primary lumen 201 to the perfusion branch lumen 231a. The perfusion outlet opening 232a allows fluid communication between the vascular prosthesis 200 and the perfusion apparatus 300 via a first quick connect feature 233, as discussed in greater detail below.

In certain embodiments, the diameter for the perfusion branch conduit 230a may be greater than the diameter of the branch conduits 230. In one specific example, the diameter for the perfusion branch conduit 230a may be about 10 mm while the diameter of the branch conduits 230 may be in the range of 6 mm to 8 mm.

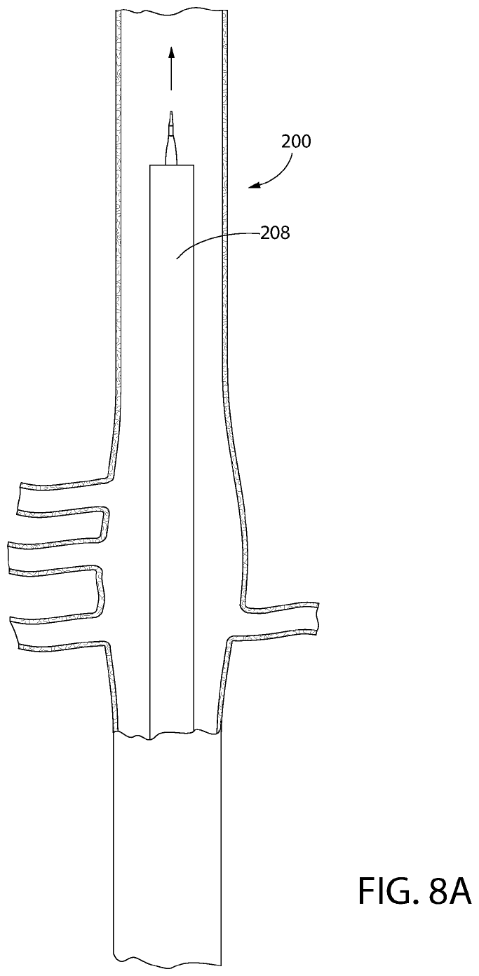

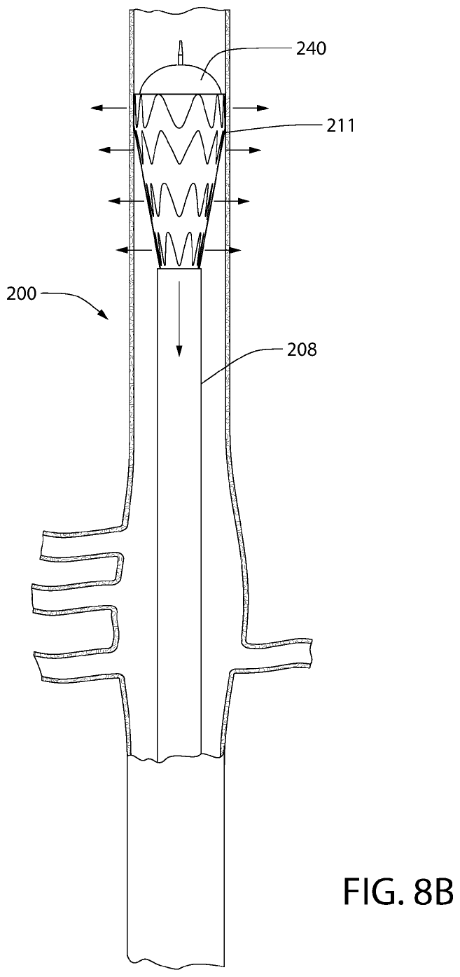

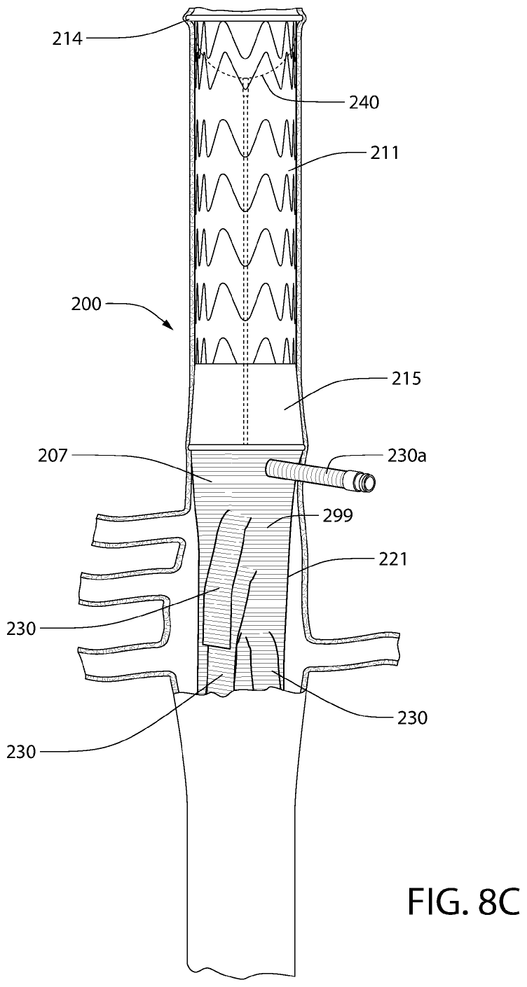

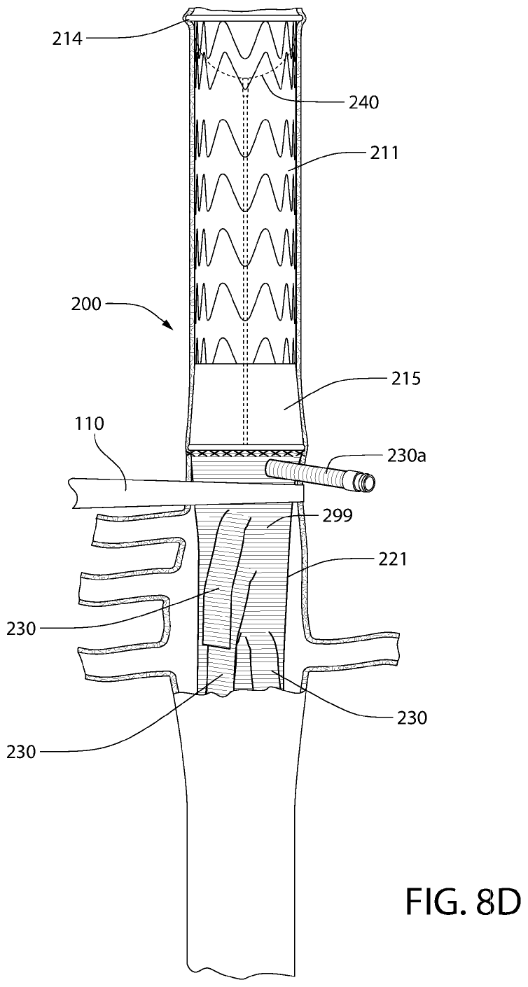

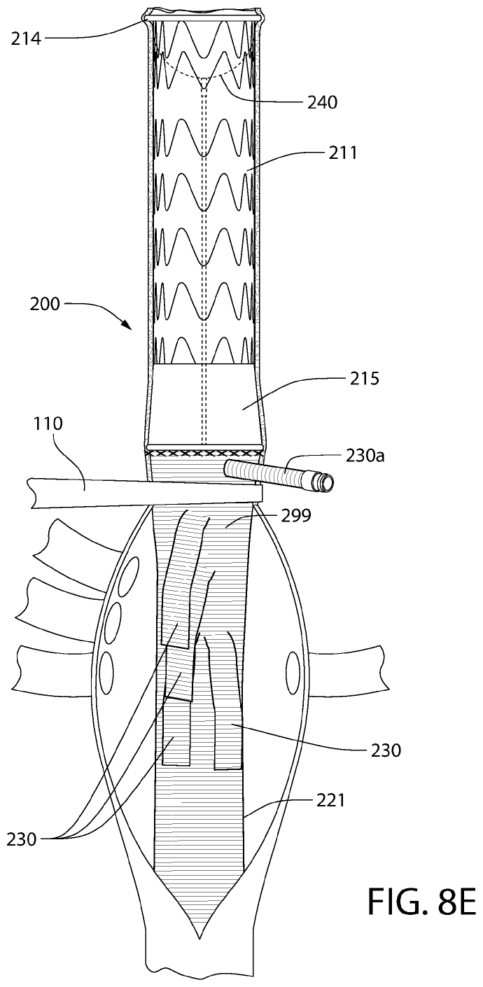

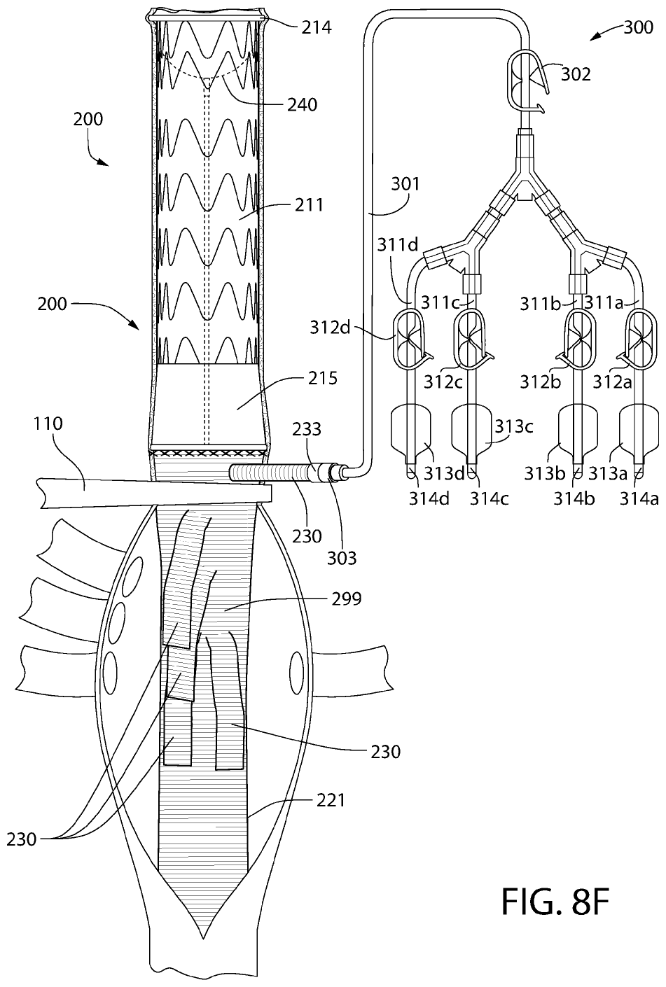

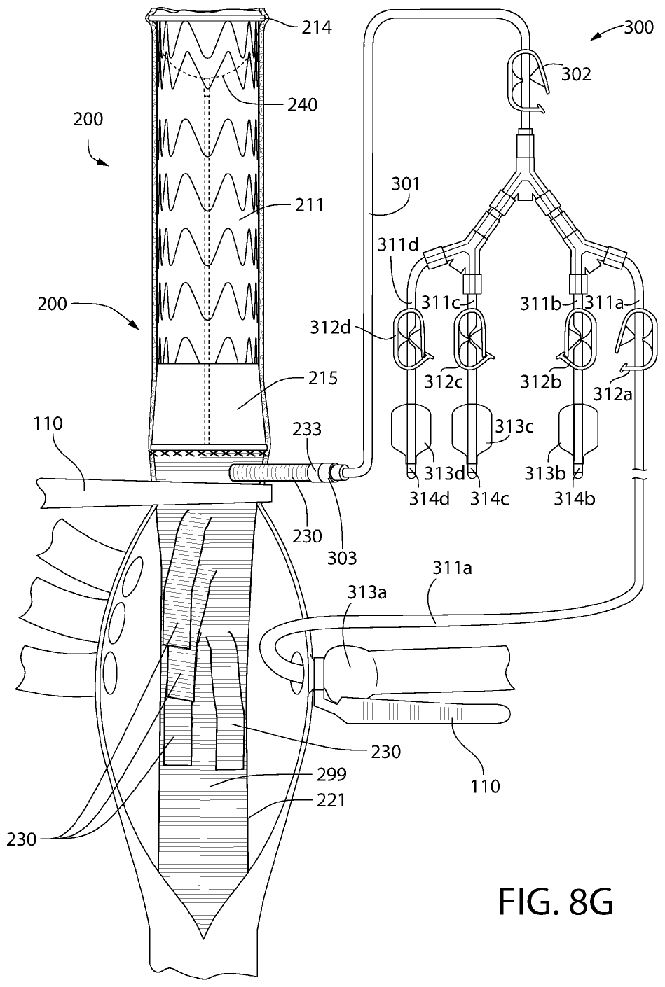

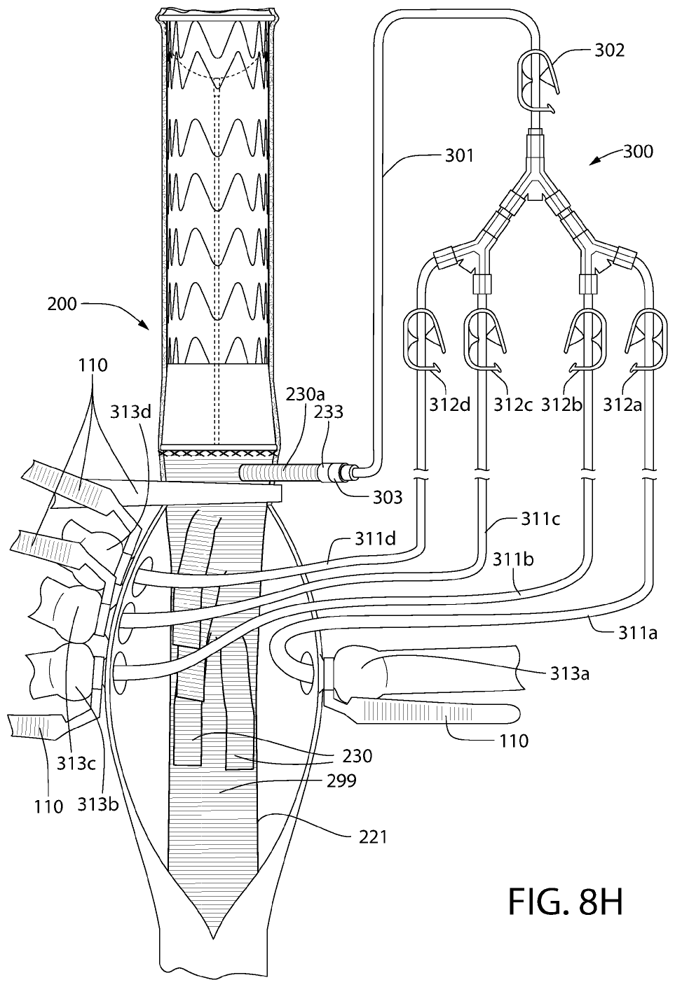

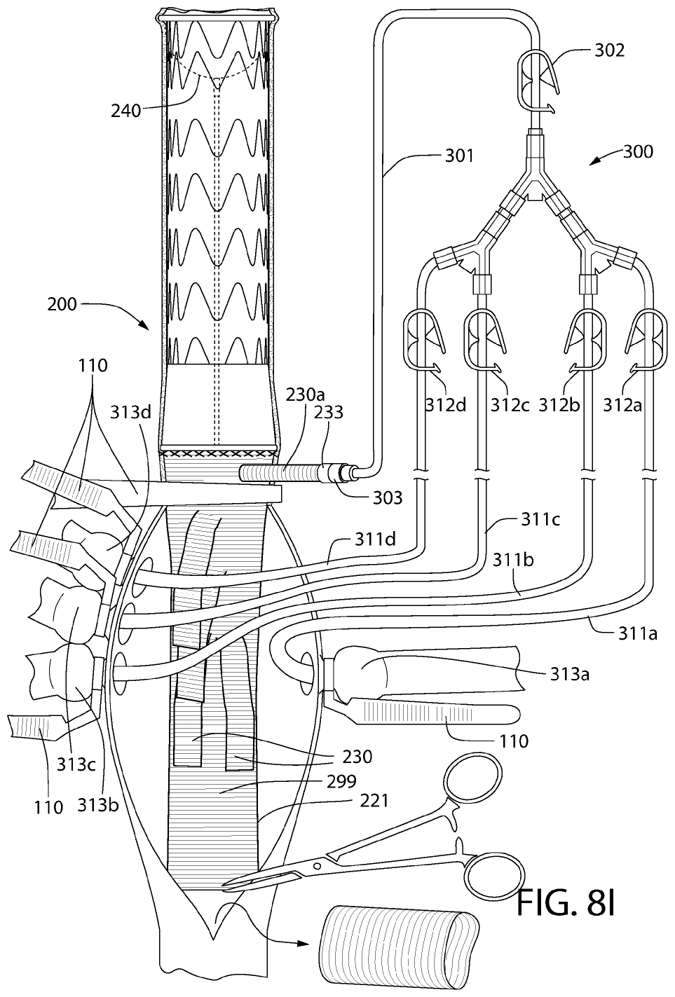

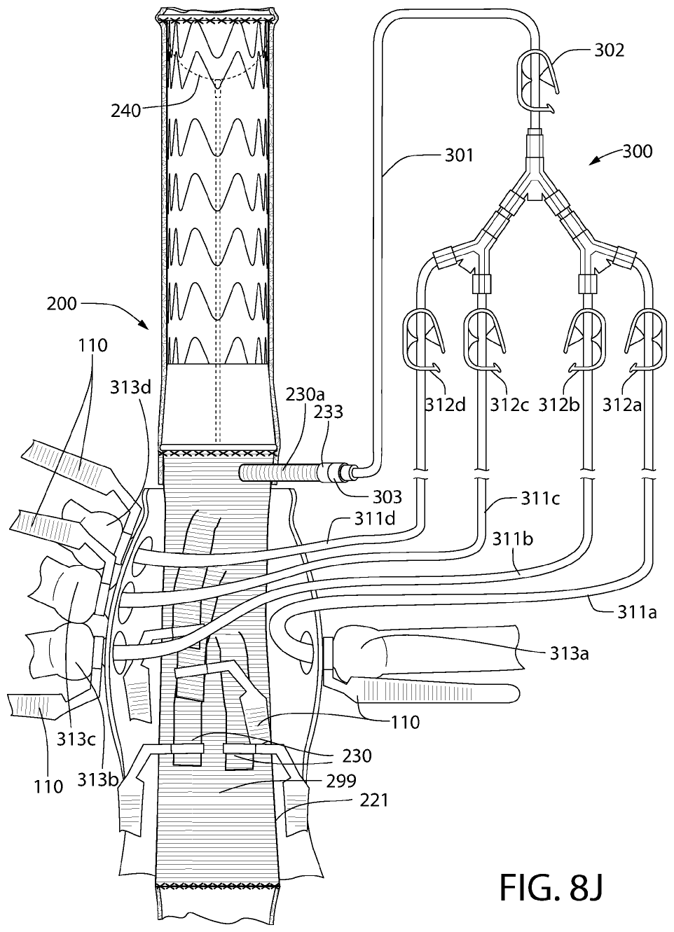

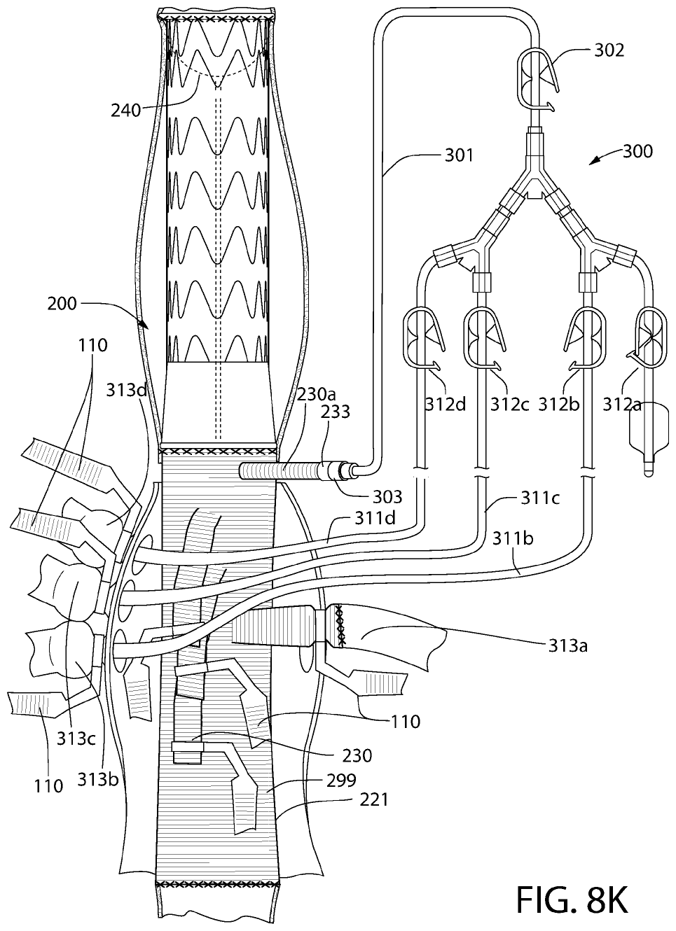

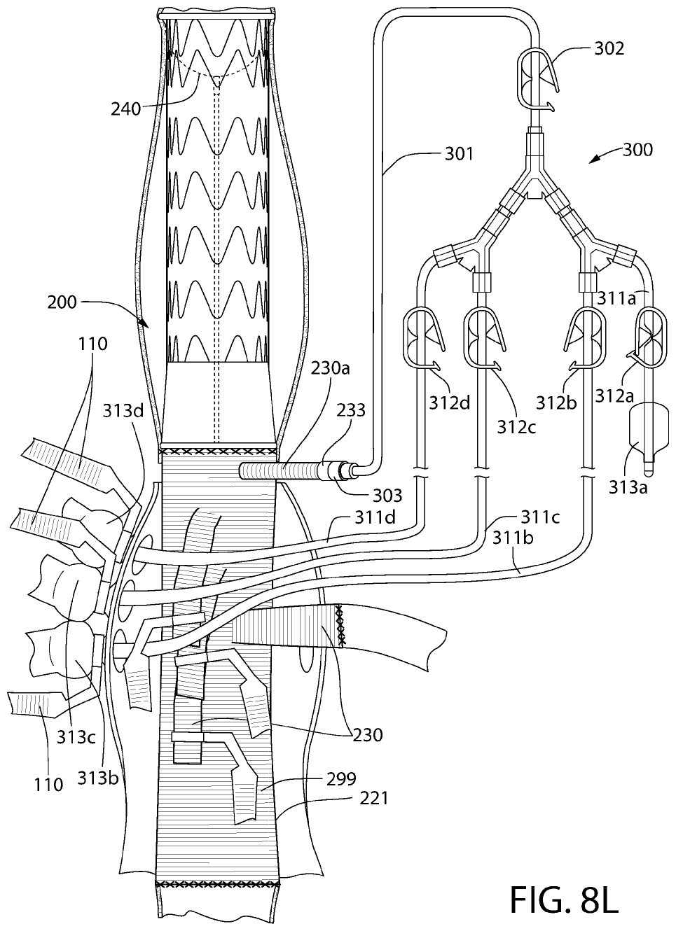

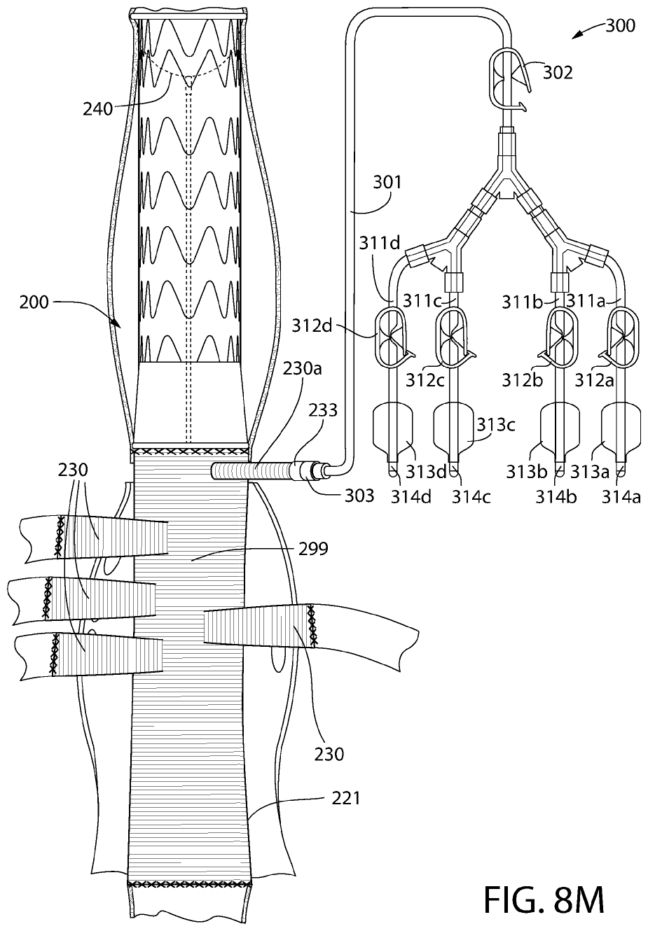

In certain embodiments of the present invention, a molding balloon 240 (see FIG. 8B) is included that is positioned within the primary lumen 201 of the prosthesis 200. The molding balloon 240 is configured to position the vascular prosthesis 200 within the aorta. In certain embodiments, the molding balloon 240 is configured so that, when inflated, the primary lumen 201 is sealed and blood is prevented from flowing from the aorta of the patient and into (or through) the primary lumen 201. When inflated, the molding balloon 240 prevents blood from flowing through the primary lumen 201 such that it would reach the branch conduits 230 or the perfusion branch conduit 230a. Although not shown, one skilled in the art would understand that the vascular prosthesis 200 may comprise additional features not shown in the drawings. For example, the vascular prosthesis 200 may further comprise a guide wire or other deployment devices which are standard in the art.

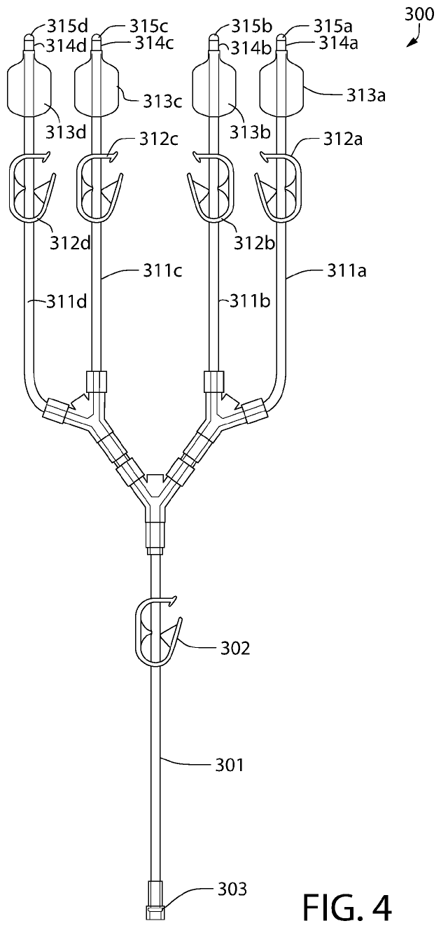

Referring to FIG. 4, the perfusion apparatus 300 will now be described. The perfusion apparatus 300 generally comprises an inlet conduit 301 and a plurality of secondary conduits 311a-d branching from the inlet conduit 301. The perfusion apparatus 300 may be made from plastic, plastic derivative, or combination thereof.

The secondary conduits 311a-d respectively comprise distal tip regions 314a-d, which includes secondary conduit outlets 315a-d. The secondary conduit outlets 315a-d allow fluid communication between the internal passageways (or lumens) of the secondary conduits 311a-d and the aortic branches of a patient when coupled thereto, such as visceral arteries. The tip portions 314a-d of the secondary conduits 311a-d may have an outer diameter that is tapered to allow easier insertion into the aortic branches of the patient. In some embodiments, the secondary conduits 311a-d respectively comprise stabilizing features 313a-d which is proximate the tip regions 314a-d. The stabilizing features 313a-d are configured to ensure a sealed fluid coupling between the passageways/lumens of the secondary conduits 311a-d and the aortic branches to which they are coupled. In some embodiments, the stabilizing feature 313 is a compressible ring-structure, such as fluid-filled balloon.

The perfusion apparatus 300 further comprises a valve assembly that allows a use to selectably allow and/or prevent the flow of fluid, such as blood, through the inlet conduit 301 and/or secondary conduits 311a-d. In the exemplified embodiment, the valve assembly comprises a valve operably coupled to the inlet conduit 301 and a valve operably coupled to each of the secondary conduits 311a-d. In the exemplified embodiment, the valve operably coupled to the inlet conduit 301 is in the form of a clamp 302 while the valves operably coupled to each of the secondary conduits 311a-d are in the form of clamps 312a-d. While the valves are exemplified as pinch clamps 302, 312a-d, other suitable type of valves can be used, such as a globe valve, a gate valve, a ball valve, a butterfly valve, a diaphragm valve, a plug valve, a needle valve, an angle valve, or combinations thereof.

The valve assembly, formed by valves 302, 312a-d, is arranged so that it can utilized to allow or prevent flow selectably through all, none, a desired singular one, or a desired combination of the secondary conduits 311a-d. As such, in the exemplified embodiment, each of the clamps 302, 312a-d can be individual altered by a user between an open flow state (in which blood can flow through the respective secondary conduit 311a-d) and a closed seal state (in which blood cannot flow through the respective secondary conduit 311a-d). As shown in FIG. 4, all of the clamps 302, 312a-d are in the open flow state.

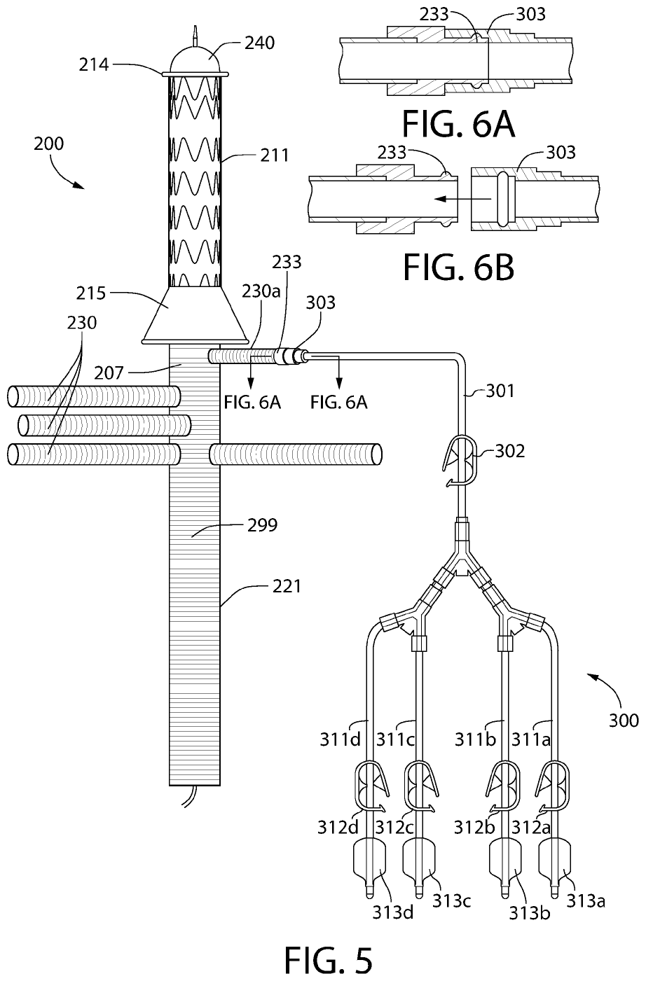

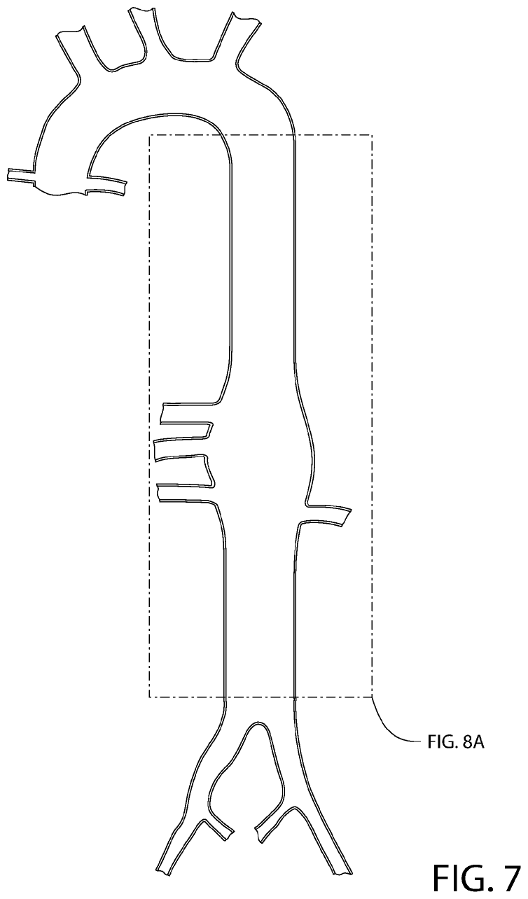

As shown in FIGS. 5 to 6B, the proximal end of the inlet conduit 301 is configured to be detachably coupled to the perfusion outlet opening 232a to form a fluid tight connection. In the exemplified embodiment, this is achieved by the presence of (and mating between) a first quick connect feature 233 on the perfusion branch conduit 230a and a second quick connect feature 303 on the inlet conduit 301. The first quick connect feature 230 may formed of material that is more rigid than the material of which the perfusion branch conduit 230a is formed, such as a plastic.

The first quick connect feature 230 may be a bayonet connector component, a threaded connector component, a snap-fit connector component, an interference fit connector component, or a combination thereof. Likewise, the second quick connect feature 303 would be the corresponding component that would be mate with the selected structure of the first quick connect feature 230. Thus, the second quick connect feature 303 would take the form of the corresponding one of a bayonet connector component, a threaded connector component, a snap-fit connector component, an interference fit connector component, or a combination thereof. When mated, the quick connect features 233, 303 fluidly couple the perfusion branch lumen 231a to the passageway/lumen of the inlet conduit 301 to supply blood thereto.

It should be note that, while the perforation apparatus 300 is shown as being separate component that is coupled to the vascular prosthesis 200, in other embodiments the perforation apparatus 300 can be permanently integrated into the vascular prosthesis 200 such that it is supplied as singular component. In such an embodiment, the

Referring to FIGS. 8A through 8I, the present application may comprise a method of repairing an aortic aneurysm using the above-described vascular prosthesis 200 and perfusion apparatus 300.

Referring to FIG. 8A, the vascular prosthesis 200 is inserted into the aorta of a patient. In a preferred embodiment, the prosthesis 200 is inserted into the descending thoracoabdominal aorta. In a more preferred embodiment, the prosthesis 200 is inserted into the descending thoracoabdominal aorta below the visceral arteries. As shown in FIGS. 8B and 8C, the sheath 208 surrounding the vascular prosthesis is removed and the prosthesis 200 is expanded so that the outer surface 205 of the vascular prosthesis 200 is in contact with the walls of the aorta. A molding balloon 240 is inflated to spatially stabilize the proximal end of the vascular prosthesis 200 against the walls of the aorta.

Referring to FIG. 8D, the prosthesis 200 may then be fluidly coupled to the aorta by attaching the hemostatic apron 215 to the walls of the aorta. In a preferred embodiment, the hemostatic apron 215 is attached to the walls of the aorta by sutures. In some embodiments, a clamp is placed in the clamping zone 207 to prevent the flow of fluid through the primary lumen 201 of the prosthesis 200. As shown in FIG. 8E, the portion of the aorta below the hemostatic apron 215 may then be opened to expose the rest of the prosthesis 200.

Referring to FIG. 8F, the inlet conduit 301 of the perfusion apparatus 300 may then be fluidly coupled to the distal end of a perfusion branch conduit 230a. The valve assembly (302, 312) of the perfusion apparatus 300 is in a closed sealed state so that fluid such as blood from the primary lumen 201 of the vascular prosthesis 200 cannot flow through the plurality of secondary conduits 311 of the perfusion apparatus 300 that branch from the inlet conduit 301.

Referring to FIGS. 8G and 8H, after the perfusion apparatus 300 is connected to the vascular prosthesis 200, one or more secondary conduits 311 may then be fluidly coupled to one or more aortic branches. In some embodiments, the secondary conduit is fluidly connected to an aortic branch by having one or more tip portions be inserted into the proximal end of one or more aortic branches, and then attached with a soft clamp. The soft clamp would be tight enough to still allow fluid to pass through to still allow fluid communication but would prevent the stabilizing feature from passing through. Once the secondary conduit is fluidly coupled to an aortic branch, the valve assembly (302, 312) is altered to an open flow state to restore blood flow to one or more aortic branches.

As shown in FIG. 8I, the distal end of the vascular prosthesis 200 may then be cut to an appropriate length that would be known by a surgeon of skill in the art, thereby forming a new distal end of the vascular prosthesis 200. As shown in, FIG. 8J, the new distal end of the vascular prosthesis 200 is then attached to a downstream portion of the aorta. In a preferred embodiment, the new distal end of the vascular prosthesis 200 is attached to the downstream portion of the aorta with sutures.

Referring to FIGS. 8J through 8M, the clamp 110 is removed from the clamping zone 207 and the one or more secondary conduits 311 are then removed from the one or more aortic branches. One or more branch conduits 230 of the vascular prosthesis 200 are then attached to one or more aortic branches. In certain embodiments, a clamp is placed at the end of each of the one or more branch conduits 230 of the vascular prosthesis 200 to prevent the flow of fluid through the branch lumen 231. When the one or more branch conduits 230 of the vascular prosthesis 200 are attached to one or more aortic branches, the clamp 110 is then removed to restore blood flow.

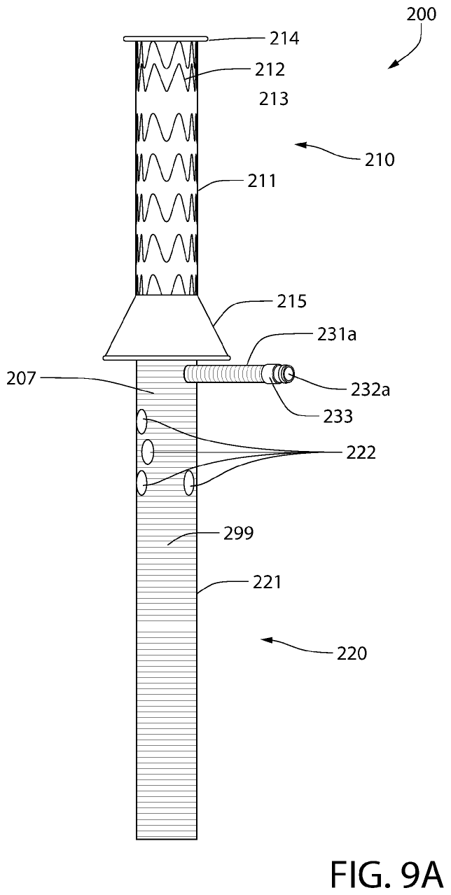

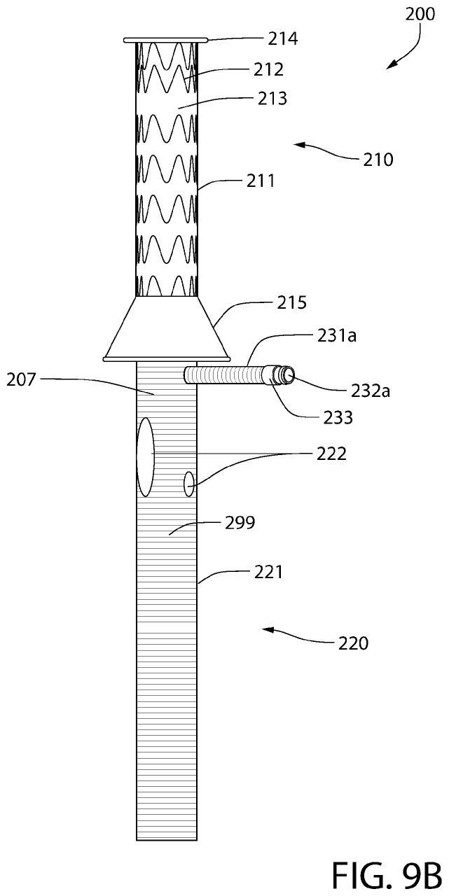

Referring now to FIGS. 9A and 9B, the vascular prosthesis 200 may be configured in a variety of alternative configurations including some embodiments without any branch conduits 230. In some embodiments, the openings 222 can be fluidly coupled directly to an aortic branch such as by using a Carrel patch. In such embodiments, one or more of the branch conduits 230 can be omitted while the associated opening 222 remains such that the opening can be directly connected to an aortic branch by using the Carrel patch. As shown in FIG. 9B, the openings 222 may be of various sizes. In such embodiments, multiple aortic branches may be directly connected to a single opening 222.

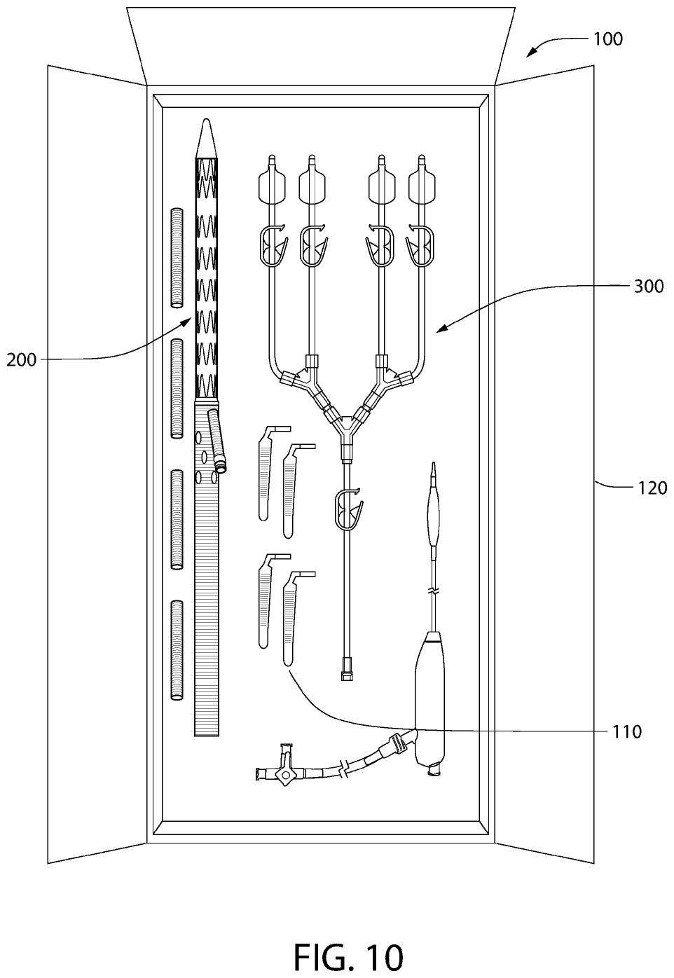

As shown in FIG. 10, the surgical kit 100 may comprise a plurality of branch conduits 230 that are detached from the vascular prosthesis. The branch conduits 230 may then be attached during surgery, as needed. In some embodiments, a plurality of aortic branches may be coupled to the vascular prosthesis using any combination of direct coupling with an opening 222 and coupling with a branch conduit 230.

While the invention or inventions have been described with respect to specific examples, those skilled in the art will appreciate that there are numerous variations and permutations of the above-described invention(s). It is to be understood that other embodiments may be utilized and structural and functional modifications may be made without departing from the scope of the present invention(s). Thus, the spirit and scope should be construed broadly as set forth in the appended claims.

### EXEMPLARY CLAIMS

Exemplary claim 1: A surgical kit for repairing an aortic aneurysm comprising: a vascular prosthesis comprising: a primary conduit forming a primary lumen extending from a primary inlet opening to a primary outlet opening, the primary conduit configured to be fluidly coupled to an aorta of a patient; and a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen; and a perfusion apparatus comprising: an inlet conduit having a proximal end configured to be fluidly coupled to the distal end of the perfusion branch conduit; a plurality of secondary conduits branching from the inlet conduit and configured for fluid coupling to aortic branches; and a valve assembly configured to selectably alter the plurality of secondary conduits between an open flow state and a closed sealed state.

Exemplary claim 2: The surgical kit according to claim 1 wherein the primary conduit of the vascular prosthesis comprises a stent-graft portion and a tube-graft portion that meet at a junction region, the stent-graft portion comprising the primary inlet opening and the tube graft portion comprising the primary outlet opening.

Exemplary claim 3: The surgical kit according to claim 2 wherein the stent-graft portion comprises a flexible biocompatible membrane attached to a support structure, the stent-graft portion configured to be capable of being radially expanded from a collapsed state to a deployed state.

Exemplary claim 4: The surgical kit according to any one of claims 1 to 3 wherein the vascular prosthesis further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

Exemplary claim 5: The surgical kit according to claim 4 wherein the hemostatic apron is a truncated cone-shaped flange that extends from a top end to bottom end, the top end of the hemostatic apron connected to the primary conduit above a junction region and the bottom end of the hemostatic apron located below the junction region.

Exemplary claim 6: The surgical kit according to any one of claims 1 to 5 wherein the vascular prosthesis further comprises a hemostatic collar protruding radially from the primary conduit.

Exemplary claim 7: The surgical kit according to claim 6 wherein the hemostatic collar comprises interspersed projections or barbs to prevent the vascular prosthesis from migrating within the aorta.

Exemplary claim 8: The surgical kit according to any one of claims 1 to 7 wherein the vascular prosthesis further comprises a plurality of branch conduits, each of the branch conduits forming a branch lumen extending from a branch inlet opening in the primary conduit to a branch outlet opening, the branch inlet opening in fluid communication with the primary lumen, and a distal end of each of the branch conduits configured to be fluidly coupled to an aortic branch;

Exemplary claim 9: The surgical kit according to claim 8 wherein the perfusion branch opening is located closer to the primary inlet opening than each and every of the branch inlet openings.

Exemplary claim 10: The surgical kit according to any one of claims 1 to 9 wherein the distal end of the perfusion branch conduit comprises a first quick connect feature and the proximal end of the inlet conduit of the perfusion apparatus comprises a second quick connect feature configured to be matable with the first quick connect feature to fluidly couple the perfusion branch lumen and a passageway of the inlet conduit.

Exemplary claim 11: The surgical kit according to claim 10 wherein the first second quick connect features are selected from a group consisting of bayonet connector component, a threaded connector component, a snap-fit connector component, an interference fit connector component, and combinations thereof.

Exemplary claim 12: The surgical kit according to any one of claims 10 to 11 wherein the perfusion branch lumen has a transverse cross-sectional area that is less than the transverse cross-sectional area of the primary lumen but greater than a transverse cross-sectional area of any of the plurality of branch lumens.

Exemplary claim 13: The surgical kit according to any one of claims 10 to 12 wherein the perfusion branch conduit comprises a main body portion extending from the primary lumen and a distal portion comprising the distal end, the distal portion having a greater rigidity than the main body portion.

Exemplary claim 14: The surgical kit according to any one of claims 1 to 13 further comprising an inflatable molding balloon configured to be positioned within the primary lumen of the primary conduit.

Exemplary claim 15: The surgical kit according to any one of claims 1 to 14 wherein each of the plurality of secondary conduits of the perfusion apparatus has a distal end configured to be fluidly coupled to one of the aortic branches.

Exemplary claim 16: The surgical kit according to claim 15 wherein, for each of the plurality of secondary conduits of the perfusion apparatus, the distal end comprises a tip portion having a tapered outer diameter.

Exemplary claim 17: The surgical kit according to any one of claims 1 to 16 wherein each of the secondary conduits of the perfusion apparatus further comprises a stabilizing feature for ensure a sealed fluid coupling between a passageways of the secondary conduit and the aortic branch.

Exemplary claim 18: The surgical kit according to claim 17 wherein the stabilizing feature comprises a compressible ring-structure.

Exemplary claim 19: The surgical kit according to any one of claims 1 to 18 wherein the perfusion apparatus comprises four of the secondary conduits.

Exemplary claim 20: The surgical kit according to any one of claims 1 to 18 wherein the valve assembly is configured to be able to individually alter each one of the plurality of secondary conduits between the open flow state and the closed sealed state independent of the other ones of the secondary conduits.

Exemplary claim 21: The surgical kit according to claim 20 wherein the valve assembly is further configured to be able to alter the inlet conduit of the perfusion apparatus between an open flow state and a closed sealed state.

Exemplary claim 22: The surgical kit according to any one of claims 20 to 21 wherein the valve assembly comprises a plurality of valves, each of the plurality of valves operably coupled to one of the plurality of secondary conduits.

Exemplary claim 23: The surgical kit according to claim 22 wherein each of plurality of valves is a clamp.

Exemplary claim 24: The surgical kit according to any one of claims 1 to 23 further comprising a sealed package containing the vascular prosthesis, the perfusion apparatus, and the valve assembly.

Exemplary claim 25: A vascular prosthesis comprising: a primary conduit forming a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; a plurality of branch conduits, each of the branch conduits forming a branch lumen extending from a branch inlet opening in the primary lumen to a branch outlet opening, the branch inlet opening in fluid communication with the primary lumen, and a distal end of each of the branch conduits configured to be fluidly coupled to an aortic branch; a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen, and the distal end of the perfusion branch conduit comprising a first quick connect feature.

Exemplary claim 26: The vascular prosthesis according to claim 25 wherein the primary conduit of the vascular prosthesis comprises a stent-graft portion and a tube-graft portion that meet at a junction region, the stent-graft portion comprising the primary inlet opening and the tube graft portion comprising the primary outlet opening.

Exemplary claim 27: The vascular prosthesis according to any one of claims 25 to 26 wherein the vascular prosthesis further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

Exemplary claim 28: The vascular prosthesis according to any one of claims 25 to 27 further comprising a hemostatic collar protruding radially from the primary conduit.

Exemplary claim 29: The vascular prosthesis according to claim 28 wherein the hemostatic collar comprises interspersed projections or barbs to prevent the vascular prosthesis from migrating within the aorta.

Exemplary claim 30: The vascular prosthesis according to any one of claims 26 to 29 further comprising a hemostatic apron protruding radially from the junction region.

Exemplary claim 31: The vascular prosthesis according to any one of claims 25 to 30 further comprising a clamping zone, wherein the clamping zone is located between the perfusion branch conduit and the plurality of branch conduits.

Exemplary claim 32: The vascular prosthesis according to any one of claims 25 to 31 wherein the first quick connect feature is selected from a group consisting of bayonet connector component, a threaded connector component, a snap-fit connector component, an interference fit connector component, and combinations thereof.

Exemplary claim 33: The vascular prosthesis according to any one of claims 25 to 32 wherein the perfusion branch opening is located closer to the primary inlet opening than each and every one of the branch inlet openings.

Exemplary claim 34: A vascular prosthesis comprising: a primary conduit comprising a primary lumen, extending from a primary inlet opening to a primary outlet opening; a plurality of branch inlet openings; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

Exemplary claim 35: The vascular prosthesis according to claim 34 wherein the primary conduit is configured so that the primary inlet opening can be fluidly coupled to a portion of an aorta upstream of an aneurysm and the primary outlet opening can be fluidly coupled to a portion of an aorta downstream of an aneurysm.

Exemplary claim 36: The vascular prosthesis according to claim 34 further comprising a stent-graft portion and a tube-graft portion that meet at a junction region, the stent-graft portion comprising the primary inlet opening and the tube graft portion comprising the primary outlet opening; and wherein the plurality of branch inlet openings are located in the tube-graft portion.

Exemplary claim 37: The vascular prosthesis according to claim 36 wherein the tube graft portion is made of a material selected from a group consisting of polyester, polyurethane, polyethylene terephthalate (PET/Dacron), polytetrafluoroethylene (PTFE), expanded polytetrafluoroethylene (ePTFE), and thermoplastic polyethylene.

Exemplary claim 38: The vascular prosthesis according to any one of claims 34 to 37 wherein the plurality of branch inlet openings comprise a first branch inlet opening and a second branch inlet opening, the first branch inlet opening being larger than the second branch inlet opening.

Exemplary claim 39: The vascular prosthesis according to any one of claims 34 to 38 further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

Exemplary claim 40: The vascular prosthesis according to claim 39 wherein the hemostatic apron is a truncated cone-shaped flange that extends from a top end to bottom end, the top end of the hemostatic apron connected to the primary conduit above a junction region and the bottom end of the hemostatic apron located below the junction region.

Exemplary claim 41: The vascular prosthesis according to any one of claims 34 to 40 wherein the vascular prosthesis further comprises a hemostatic collar protruding radially from the primary conduit.

Exemplary claim 42: The vascular prosthesis according to claim 41 wherein the hemostatic collar comprises interspersed projections or barbs to prevent the vascular prosthesis from migrating within the aorta.

Exemplary claim 43: A vascular prosthesis comprising: a primary conduit comprising a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; four branch conduits, each of the four branch conduits forming a branch lumen extending from a branch inlet opening in the primary conduit to a branch outlet opening, the branch inlet opening in fluid communication with the primary lumen, and a distal end of each of the four branch conduits configured to be fluidly coupled to an aortic branch; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

Exemplary claim 44: A method of repairing an aortic aneurysm, the method comprising: fluidly coupling: (i) a proximal end of a primary conduit of a vascular prosthesis to a first portion of an aorta of a patient upstream of the aortic aneurysm; and (ii) a distal end of the primary conduit of the vascular prosthesis to a second portion of the aorta downstream of the aortic aneurysm, wherein one or more aortic branches of the patient are located between the first and second portions of the aorta, thereby allowing blood to flow through a primary lumen of the primary conduit; fluidly coupling an inlet conduit of a perfusion apparatus to a distal end of a perfusion branch conduit of the vascular prosthesis, a valve assembly being in a closed sealed state such that blood from the primary lumen of the vascular prosthesis cannot flow through a plurality of secondary conduits of the perfusion apparatus that branch from the inlet conduit; fluidly coupling distal ends of the secondary conduits to the one or more aortic branches; and altering the valve assembly to an open flow state to restore blood flow to the one or more aortic branches.

1. A surgical kit for repairing an aortic aneurysm comprising:
a vascular prosthesis comprising:
a primary conduit forming a primary lumen extending from a primary inlet opening to a primary outlet opening, the primary conduit configured to be fluidly coupled to an aorta of a patient; and
a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen; and
a perfusion apparatus comprising:
an inlet conduit having a proximal end configured to be fluidly coupled to the distal end of the perfusion branch conduit;
a plurality of secondary conduits branching from the inlet conduit and configured for fluid coupling to aortic branches; and
a valve assembly configured to selectably alter the plurality of secondary conduits between an open flow state and a closed sealed state.

2. The surgical kit according to claim 1 wherein the primary conduit of the vascular prosthesis comprises a stent-graft portion and a tube-graft portion that meet at a junction region, the stent-graft portion comprising the primary inlet opening and the tube graft portion comprising the primary outlet opening.

3. The surgical kit according to claim 2 wherein the stent-graft portion comprises a flexible biocompatible membrane attached to a support structure, the stent-graft portion configured to be capable of being radially expanded from a collapsed state to a deployed state.

4. The surgical kit according to claim 1 wherein the vascular prosthesis further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

5. The surgical kit according to claim 4 wherein the hemostatic apron is a truncated cone-shaped flange that extends from a top end to bottom end, the top end of the hemostatic apron connected to the primary conduit above a junction region and the bottom end of the hemostatic apron located below the junction region.

6. The surgical kit according to claim 1 wherein the vascular prosthesis further comprises a hemostatic collar protruding radially from the primary conduit.

7. The surgical kit according to claim 6 wherein the hemostatic collar comprises interspersed projections or barbs to prevent the vascular prosthesis from migrating within the aorta.

8.-24. (canceled)

25. A vascular prosthesis comprising:
a primary conduit forming a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm;
a plurality of branch conduits, each of the branch conduits forming a branch lumen extending from a branch inlet opening in the primary lumen to a branch outlet opening, the branch inlet opening in fluid communication with the primary lumen, and a distal end of each of the branch conduits configured to be fluidly coupled to an aortic branch;
a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen, and the distal end of the perfusion branch conduit comprising a first quick connect feature.

26. The vascular prosthesis according to claim 25 wherein the primary conduit of the vascular prosthesis comprises a stent-graft portion and a tube-graft portion that meet at a junction region, the stent-graft portion comprising the primary inlet opening and the tube graft portion comprising the primary outlet opening.

27. The vascular prosthesis according to claim 25 wherein the vascular prosthesis further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

28. The vascular prosthesis according to claim 25 further comprising a hemostatic collar protruding radially from the primary conduit.

29. The vascular prosthesis according to claim 28 wherein the hemostatic collar comprises interspersed projections or barbs to prevent the vascular prosthesis from migrating within the aorta.

30. The vascular prosthesis according to claim 26 further comprising a hemostatic apron protruding radially from the junction region.

31. The vascular prosthesis according to claim 25 further comprising a clamping zone, wherein the clamping zone is located between the perfusion branch conduit and the plurality of branch conduits.

32. (canceled)

33. (canceled)

34. A vascular prosthesis comprising:
a primary conduit comprising a primary lumen, extending from a primary inlet opening to a primary outlet opening;
a plurality of branch inlet openings;
a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

35. The vascular prosthesis according to claim 34 wherein the primary conduit is configured so that the primary inlet opening can be fluidly coupled to a portion of an aorta upstream of an aneurysm and the primary outlet opening can be fluidly coupled to a portion of an aorta downstream of an aneurysm.

36. The vascular prosthesis according to claim 34 further comprising a stent-graft portion and a tube-graft portion that meet at a junction region, the stent-graft portion comprising the primary inlet opening and the tube graft portion comprising the primary outlet opening; and wherein the plurality of branch inlet openings are located in the tube-graft portion.

37. The vascular prosthesis according to claim 36 wherein the tube graft portion is made of a material selected from a group consisting of polyester, polyurethane, polyethylene terephthalate (PET/Dacron), polytetrafluoroethylene (PTFE), expanded polytetrafluoroethylene (ePTFE), and thermoplastic polyethylene.

38. The vascular prosthesis according to claim 34 wherein the plurality of branch inlet openings comprise a first branch inlet opening and a second branch inlet opening, the first branch inlet opening being larger than the second branch inlet opening.

39. The vascular prosthesis according to claim 34 further comprising a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

40.-44. (canceled)
